(19) United States
(12) Patent Application Publication
(10) Pub. No.: US 2013/0035257 A1
(43) Pub. Date: Feb. 7, 2013

(54) MICROFLUIDIC ARRAYS AND METHODS FOR THEIR PREPARATION AND USE

(71) Applicants: Linfen YU, Vancouver (CA); Karen C. CHEUNG, Vancouver (CA)

(72) Inventors: Linfen YU, Vancouver (CA); Karen C. CHEUNG, Vancouver (CA)

(73) Assignee: UNIVERSITY OF BRITISH COLUMBIA, Vancouver (CA)

(21) Appl. No.: 13/567,801

(22) Filed: Sep. 10, 2012

Related U.S. Application Data
(60) Provisional application No. 61/515,349, filed on Aug. 5, 2011.

(51) Int. Cl. C40B 30/08; B32B 37/14; C40B 40/02; C12N 11/02; C40B 50/06; C40B 60/08

(52) U.S. Cl. 506/11; 506/26; 506/37; 506/14; 435/177; 156/245

(57) ABSTRACT

Methods of isolating at least one cell of interest, methods of making fixed arrays, arrays comprising a glass substrate bonded to a patterned siloxane structure having inlets, outlets and microchannels, array kits, and methods of making microfluidic apparati are provided in the present application.

### CROSS-REFERENCE TO RELATED APPLICATIONS

This application claims benefit of priority to U.S. Provisional Patent Application No. 61/515,349 filed on Aug. 5, 2011, the entire contents of which are hereby incorporated by reference.

### BACKGROUND

Microfluidic systems have already found many applications in different stages of the drug discovery and drug development processes, including sample pre-concentration, separations, protein arrays, cellular interaction arrays, and cell-based high content screening. Three-dimensional (3-D) culture methods are used to study drug penetration in tumors, and multicellular tumor spheroids have received a great deal of attention in cancer research. Conventional techniques to form tumor spheroids, include growth on non-adherent surfaces or suspension in spinning flasks. However, the cells should still be transferred to a separated platform for cytotoxicity testing.

Hydrogels, which create a three-dimensional environment, are porous polymer networks. Hydrogels allow the transport of nutrients and waste away from embedded cells, and the gel network can also include specific adhesive properties for cell attachment. In cell-based drug screening, the different cellular responses exhibited in traditional 2-D monolayer versus 3-D culture have a crucial impact in the pharmacological response to drugs, which may differ between cells in 2-D and 3-D culture.

### SUMMARY OF THE INVENTION

The present application provides methods of isolating a cell of interest. In some embodiments, the methods comprise disposing a collection of hydrogel encapsulated cells on a surface to prepare a fixed array, assaying the array to identify at least one hydrogel encapsulated cell of interest, and removing the at least one hydrogel encapsulated cell of interest from the array to provide an isolated hydrogel encapsulated cell.

The present application also provides methods of making fixed arrays of cells. In some embodiments, the methods comprise mixing alginate precursor and at least one cell in an immiscible solvent to form a dispersed phase, gelling the dispersed phase using calcium ions to form at least one alginate encapsulated cell, and disposing the alginate encapsulated cell onto a surface to prepare a fixed array.

The present application also provides an array for cells, having in some embodiments a glass substrate bonded to a patterned siloxane structure having inlets, outlets and microchannels.

The present application provides an array kit. In some embodiments, the array kit comprises a glass substrate and a patterned siloxane structure having microchannels, inlets and outlets.

The present application also provides another array kit. In some embodiments, the array kit comprises a glass substrate; a cell culture mold comprising microchannels, inlets and outlets; a droplet formation mold having at least one channel and a nozzle; and a siloxane substrate.

The present application provides methods of method of making a microfluidic apparatus. In some embodiments, the methods comprise applying a layer of photoresist to a silicon substrate to make a silicon mold, pouring a layer of siloxane into the silicon mold to make a patterned siloxane structure, bonding the patterned siloxane structure to a glass substrate to form a cell culture structure, forming a droplet formation mold comprising at least one channel and a nozzle, pouring a layer of siloxane into the droplet formation mold to make a siloxane droplet formation structure, and bonding the siloxane droplet formation structure to a siloxane substrate to form a droplet formation structure.

The present application further provides methods of making a fixed array of cells. In some embodiments, the methods comprise incubating a cell suspended in a hydrogel in a buffer or medium to form a hydrogel encapsulated cell, and disposing the hydrogel encapsulated cell onto a surface to prepare a fixed array.

### DESCRIPTION OF DRAWINGS

FIG. 1 shows a top view of a cell culture microfluidic chip according to one embodiment.

FIG. 2 shows a side view of a cell culture microfluidic chip according to FIG. 1.

FIG. 3 shows a droplet formation microfluidic chip according to one embodiment.

FIG. 4 depicts droplet formation within a microfluidic chip according to FIG. 3.

FIG. 5 depicts alginate beads trapped in the micro sieves of FIG. 1.

FIG. 6 shows the distribution of alginate droplet diameter for alginate beads produced by microsieves of FIG. 1.

FIG. 7A depicts encapsulated dispersed cells within alginate beads according to one embodiment.

FIG. 7B depicts spheroids of cells according to one embodiment.

FIG. 8 shows images of LCC6/Her2 breast tumor cells proliferating and forming multicellular spheroids while encapsulated in alginate beads according to one embodiment.

FIG. 9 provides a chart showing effects of doxorubicin concentration on cell survival rate in various culture environments according to one embodiment.

FIG. 10 provides a chart showing effects of doxorubicin concentration on cell survival rate before and after treatment according to one embodiment.

### DETAILED DESCRIPTION

The above summary of the present application is not intended to describe each illustrated embodiment or every possible implementation of the present application. The detailed description, which follows, particularly exemplifies these embodiments.

Before the present compositions and methods are described, it is to be understood that they are not limited to the particular compositions, methodologies or protocols described, as these may vary. It is also to be understood that the terminology used in the description is for the purpose of describing the particular versions or embodiments only, and is not intended to limit their scope which will be limited only by the appended claims.

It must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

“Optional” or “optionally” of “may” means that the subsequently described event or circumstance may or may not occur, and that the description includes instances where the event occurs and instances where it does not.

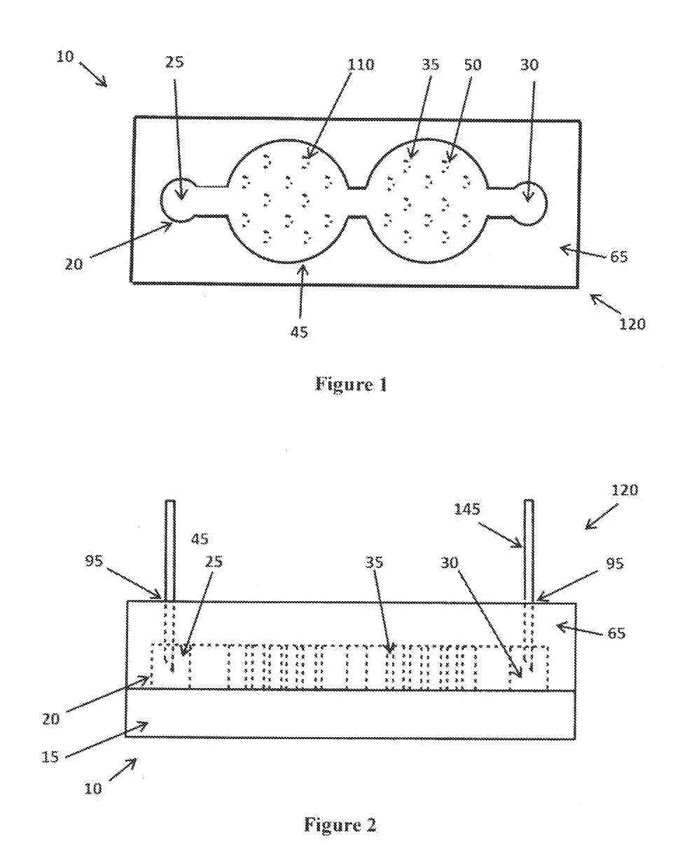

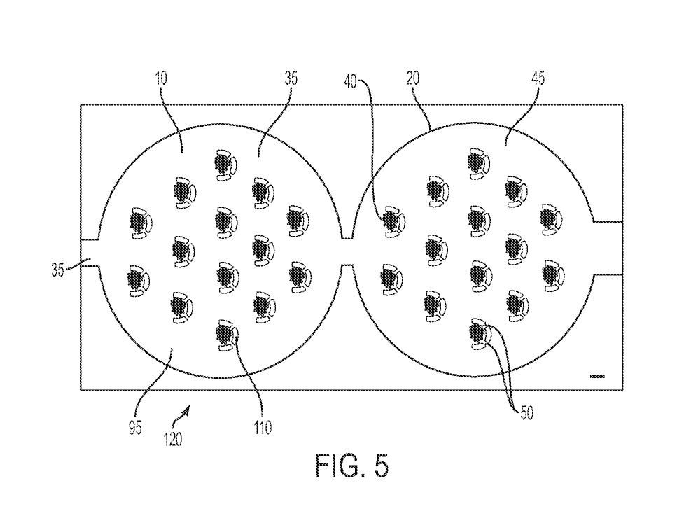

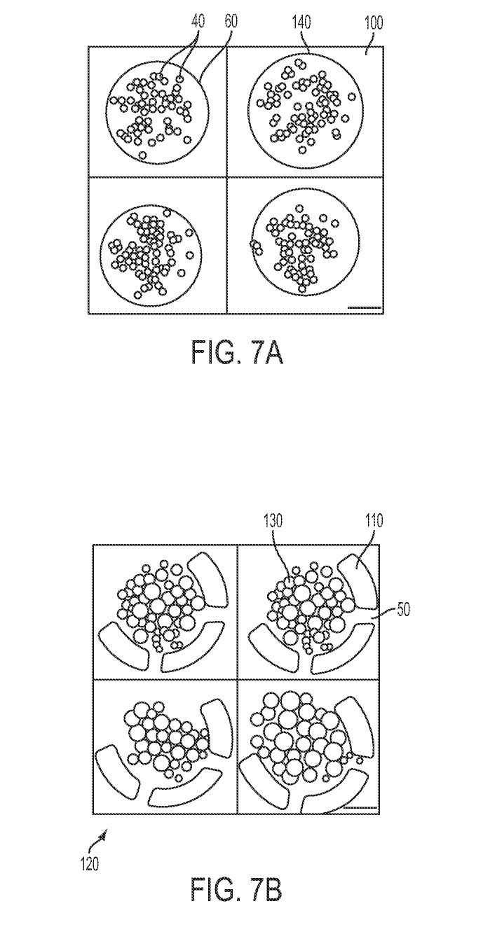

The present application provides for an array 10. The array 10 is comprised of a glass substrate 15 bonded to a patterned siloxane structure 20 having inlets 25, outlets 30, and microchannels 35 (FIGS. 1 and 2). The inlet 25 provides access to the microchannel 35 so that fluids can go into the channel(s). The outlet 30 provides access to the microchannel 35 so that fluids can exit the channel(s). The microchannels 35 are connected to their inlets 25 and outlets 30. Inlets 25 are placed at one end of the microchannels 35 and outlets 30 are placed at the other end. Diameters of the inlets 25 and outlets 30 are typically on the order of several hundred microns. Microchannels 35 typically range from tens to hundreds of microns in height and width, and from hundreds of microns to millimeters in length. In some embodiments, the patterned siloxane structure 20 comprises at least one chamber 45 having the microchannels 35. In other embodiments, the microchannels 35 comprise sieves 110, weirs, cavities, or wells, or any combination thereof. In some embodiments, the patterned siloxane structure 20 comprises at least one aperture 50 to facilitate trapping of an alginate encapsulated cell 40. The patterned siloxane structure 20 may comprise a material selected from poly-(dimethylsiloxane), polyurethane, polystyrene, parylene, and polyimide, or any combination thereof. In some embodiments, the patterned siloxane structure 20 is transparent. In some embodiments, the array 10 is further comprised of a collection of alginate encapsulated cells 40 trapped in the microchannel sieves 110 (FIGS. 5 and 7).

Some embodiments include a cell culture microfluidic chip 120. A cell culture microfluidic chip has an array 10, at least one inlet 25 and an outlet 30. In some embodiments, alginate beads 100 are introduced by a needle 145 through a hole 95 in the siloxane substrate 65 into the inlet 25. The alginate beads 100 flow through the microchannel 35 and is captured on a microsieve 110 having apertures 50 to allow fluid displacement. The medium flow is fed from the inlet 25 to the outlet 30 where it exits through a hole 95 and a needle 145.

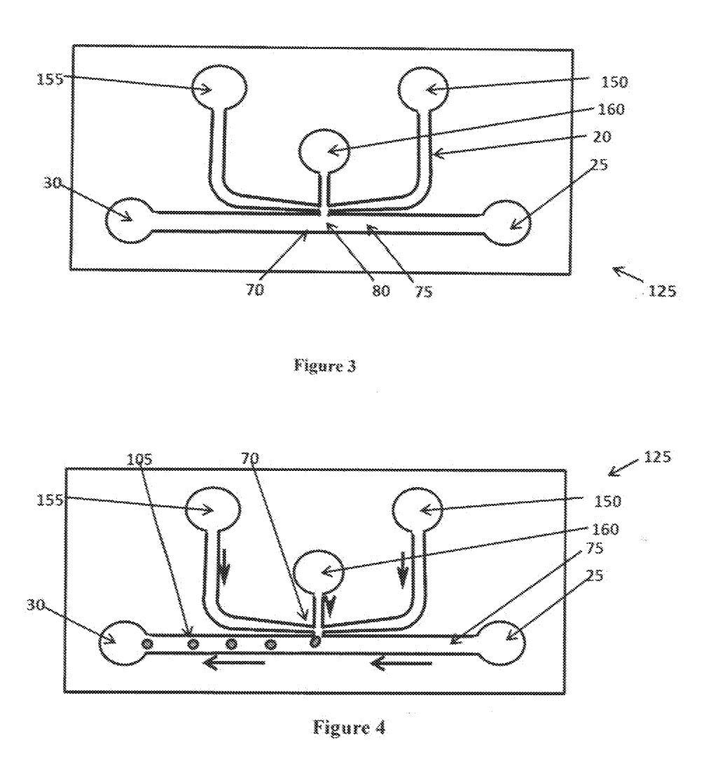

The patent application provides for a droplet formation chip 125 (FIGS. 3 and 4). A droplet formation chip 125 has an inlet 25, at least one channel 75 and an outlet 30. The siloxane droplet formation chip 125 has a droplet formation structure 70 having a nozzle 80. In some embodiments, droplets 105 are formed at the nozzle 80 by the mixing of oil from oil inlet 155, medium from medium inlet 150 and a mixture of alginate and cells from alginate/cell inlet 160. In embodiments, the droplets 105 formed are swept from the nozzle 80 by the flow of oil from the inlet 25 to the outlet 30. Droplets of one fluid (dispersed phase—here, alginate, cells, and medium) are formed within another fluid (continuous phase—here, oil). The size of the nozzle (“orifice”) has a strong influence on the size of the droplets which are formed. The nozzle is placed relatively close to the inlets. After droplet formation, the droplets flow downstream. The geometry described here is a T-junction configuration. The droplet formation structure 70 may also be a shear-focusing geometry. Specific examples of the channel 75 and nozzle orifice 80 heights and widths are independently 10 microns, 20 microns, 30 microns, 40 microns, 50 microns, 75 microns, 100 microns, 200 microns, 300 microns, 500 microns, 1000 microns, 1500 microns, or range between any two of these values.

The present application also provides for an array kit. The array kit comprises a glass substrate 15 and a patterned structure 20 having inlets 25, outlets 30, and microchannels 35. In embodiments, the array kit further comprises a hydrogel 60 to encapsulate cells. The hydrogel 60 may be selected from alginate, collagen, and Matrigel™, or any combination thereof. In embodiments of the array kit, the patterned siloxane structure 20 comprises a material selected from poly-(dimethylsiloxane), polyurethane, polystyrene, parylene, and polyimide, or any combination thereof. In various embodiments, the patterned siloxane structure 20 is transparent. In some embodiments, the array kit comprises a siloxane substrate 65. In various embodiments the array kit comprises a siloxane droplet formation chip 125 having at least one channel 75 and a nozzle 80.

The application further provides for an array kit comprising a cell culture device 120 comprising inlets 25, outlets 30, and microchannels 35, and a droplet formation device 125 having at least one channel 75 and a nozzle 80, and a siloxane substrate 65. The siloxane substrate structure 20 may comprise a material selected from poly-(dimethylsiloxane), polyurethane, polystyrene, parylene, and polyimide, or any combination thereof.

The present application provides alginate to encapsulate the tumor cells and permits the formation of spheroids, while at the same time protecting the cells from shear during the perfusion of culture medium. In contrast to Matrigel or collagen, alginate can be easily de-cross-linked in the presence of a chelator, and the released cells can be harvested for further assays.

The present application provide methods for identifying and optionally isolating at least one cell of interest. In some embodiments, the method comprises disposing a collection of hydrogel encapsulated cells on a surface 95 to prepare a fixed array, and assaying the array to identify at least one hydrogel encapsulated cell of interest 40. In some embodiments, the method further comprises removing the at least one hydrogel encapsulated cell of interest 40 from the array to provide an isolated hydrogel encapsulated cell 40. The cell of interest may be selected from a tumor cell, cancer stem cell, epithelial cell, diseased cell, and normal cell, or may be more than one cell selected from any combination thereof. In some embodiments, the surface 95 is a microfluidic chip. In other embodiments, the method further comprises incubating the fixed array.

Embodiments include the collection of hydrogel encapsulated cells 40 comprising a hydrogel 60 selected from alginate, collagen, and Matrigel, or any combination thereof. Matrigel is a trade name for a gelatinous protein mixture secreted by Engelbreth-Holm-Swarm (EHS) mouse sarcoma cells. Matrigel is marketed by BD Biociences and by Trevigen Inc. under the name Cultrex BME. Embodiments include a collection of hydrogel encapsulated cells 40 comprising a hydrogel 60 selected from alginate, collagen, and Matrigel, or any combination thereof, wherein providing a collection of alginate encapsulated cells 40 comprises mixing alginate precursor and at least one cell in an immiscible solvent to form a dispersed phase and gelling the dispersed phase using a calcium ion bath to provide the collection of alginate encapsulated cells. A calcium ion bath may include calcium ions (Ca²⁺), barium ions (Ba²⁺), strontium ions (Sr²⁺), or any combination thereof. Further embodiments have the immiscible solvent selected from, for example, hexadecane, dodecane, toluene, benzene, decalin, octanol, silicone oil, vegetable oil, and fluorinated oil, or any combination thereof. Releasing the isolated hydrogel encapsulated cell may be by a chelator or a protease, or a combination thereof. Embodiments include a collection of hydrogel encapsulated cells 40 comprising a hydrogel 60 selected from alginate, collagen, and Matrigel, or any combination thereof, wherein releasing an isolated alginate encapsulated cell 40 comprises de-crosslinking the alginate using a chelator. Chelators may be selected from, for example, 2,2′-bipyridyl, dimercaptopropanol, ethylenediaminotetraacetic acid (EDTA), ethylene glycol-bis-(2-aminoethyl)-N,N,N′,N′-tetraacetic acid (EGTA), ionophores, nitrilotriacetic acid, NTA ortho-phenanthroline, gramicidin, monensin, valinomycin, salicylic acid, triethanolamine (TEA), polysaccharides, organic acids with at least two coordination groups, lipids, steroids, amino acids, peptides, phosphates, phosphonates, nucleotides, tetrapyrrols, ferrioxamines, and phenolics, or any combination thereof. Other embodiments include a collection of hydrogel encapsulated cells 40 comprising a hydrogel 60 selected from alginate, collagen, and Matrigel, or any combination thereof, wherein releasing the isolated hydrogel encapsulated cell comprises using a protease. Proteases may be selected from, for example, dispase, trypsin, chymotrypsin, elastase, cathepsins, bromelain, actimidain, calpain, caspase, papain, mir1-CP, chymosin, rennin, pepsin, plasmepsin, nepenthesin, and collagenase, or any combination thereof.

An embodiment further comprises the step of releasing the isolated hydrogel encapsulated cell to form a released non-encapsulated cell. An embodiment comprises releasing the hydrogel encapsulated cell to form a released non-encapsulated cell, then harvesting the released non-encapsulated cell. An embodiment comprises releasing the hydrogel encapsulated cell 40 to form a released non-encapsulated cell, then culturing the released non-encapsulated cell.

The present application also provides methods of making a fixed array, the method comprising mixing alginate precursor and at least one cell in an immiscible solvent to form a dispersed phase, gelling the dispersed phase using calcium salts to form at least one alginate encapsulated cell, and disposing the alginate encapsulated cell onto a surface to prepare a fixed array. In various embodiments, the immiscible solvent is selected from, for example, hexadecane, dodecane, toluene, benzene, decalin, octanol, silicone oil, vegetable oil, and fluorinated oil, or any combination thereof. In other embodiments, the method further comprises allowing the cell to proliferate within the alginate encapsulated gel. In still other embodiments, the method comprises culturing the at least one cell before mixing with the alginate precursor.

In some embodiment, alginate precursor is mixed with at least one cell in an immiscible solvent to form a dispersed phase, gelling the dispersed phase using calcium salts to form at least one alginate encapsulated cell, washing the alginate encapsulated cell, and disposing the alginate encapsulated cell onto a surface to prepare a fixed array. Embodiments include centrifuging the washed alginate encapsulated cell before disposing the cell. Still other embodiments include suspending the centrifuged alginate encapsulated cell.

The present application also provides methods of making a fixed array, the method comprising incubating a cell suspended in a hydrogel in a buffer or medium to form a hydrogel encapsulated cell, and disposing the hydrogel encapsulated cell onto a surface to prepare a fixed array. In some embodiments, the hydrogel is collagen or Matrigel™, or a combination thereof. In other embodiments the suspended cell is incubated at a temperature of at least about 25° C. In still other embodiments, the cell is suspended in hydrogel at a temperature of less than about 25° C.

The present application also provides methods of making a microfluidic apparatus, the method comprising applying a layer of photoresist to a silicon substrate to make a mold, pouring a layer of siloxane into the mold to make a patterned siloxane structure 20, bonding the patterned siloxane structure 20 to a glass substrate 15 to form a cell culture structure, forming a droplet formation mold comprising at least one main channel 75 and a nozzle 80, pouring a layer of siloxane into the droplet formation mold to make a siloxane droplet formation structure 70, and bonding the siloxane droplet formation structure 70 to a siloxane substrate 65 to form a droplet formation structure. In some embodiments, the method further comprises curing the patterned siloxane structure 20 before bonding. In other embodiments, the method further comprises curing the siloxane droplet formation structure 70 before bonding. In still other embodiments, the cell structure may comprise one or more sieves, weirs, cavities, or wells, or any combination thereof. In some embodiments, the method further comprises treating the cell culture structure in ozone or air plasma to achieve strong bonding between the glass substrate 15 and the patterned siloxane structure 20.

In some embodiments, the patterned siloxane structure 20 comprises microchannels 35, inlets 25 and outlets 30. In other embodiments, the patterned siloxane structure 20 comprises microchannels 35, inlets 25 and outlets 30; the method further comprises making holes 95 in the microfluidic apparatus to allow access to the inlets 25 and outlets 30.

In some embodiments, the alginate—encapsulated LCC6/Her2 breast tumor cells, for example, may be trapped in the microchannel 35 on sieves 110 as U-shaped sites on a microfluidic chip for long-term on-chip culture. The tumor cells may be allowed to proliferate within the alginate gel beads 100 for several days in order to form multicellular spheroids using a perfusion system. Multicellular spheroids may be used in the study of drug response. After multicellular spheroid formation, cytotoxicity assays on the spheroids may be performed by loading a drug via the same perfusion system. In some embodiments the drug is an anticancer agent. The anticancer agent may be doxorubicin. In contrast to other art in which cells may be encapsulated in beads which are maintained in suspension in a culture flask, here, the location of each alginate gel bead 100 may be maintained in the same position throughout the device seeding process, cell proliferation and spheroid formation, treatment with drug, and imaging. This system, by combining a platform for three-dimensional cell culture with precise positioning, allows an examination of the resistance of multicellular spheroids compared to standard monolayer culture at various concentrations of doxorubicin in a convenient platform which may be adapted for eventual high throughput image-based drug screening.

The combination of a microfluidic platform as well as high sensitivity fluorescence-based assays permits many simultaneous assays on tumor biopsies, from which as few as a few thousand cells are collected. The microfluidic technology will enable different drugs and drug combinations to be tested on this small sample, so that the most effective treatment for a specific patient can be identified.

The drug response over time in a single spheroid can be monitored. The device can be mounted on an automated image-capture stage for eventual high-throughput image-based drug screening. Commercially available automated cell imagers may be programmed to automatically acquire images from pre-specified locations on a motorized platform within temperature-controlled environments. These systems, such as the IN Cell 3000 (GE Healthcare), can also have confocal capability and data analysis tools for high-content screening. In this way, individual spheroids can be tracked and any spheroid subgroups with specific responses can be identified.

In the various embodiments, the on-chip tumor cell cultures may be tracked for cell viability for several days after drug treatment has ended in order to assess whether there is delay in measured cytotoxicity using dye exclusion assays such as the Live/Dead stains. Embodiments of methods allow for tracking of dependent effects on larger spheroids to investigate whether viable cells remain at the periphery while apoptotic cells concentrate at the core of the spheroids. Other embodiments utilizing large spheroids may have fixation and other staining methods to ensure the reagents can reach the spheroid core for uniform cell staining throughout the aggregate. Embodiments may use alternate stains for studies using cells which express the multidrug resistance protein MDR1 or the multidrug resistance-associated protein MRP1, since those cells actively pump out calcein-AM. Other embodiments include comparing effects of oxygen and drug gradients on spheroid size for their effect on toxicity.

One of the challenges in comparing the toxicity in multicellular aggregates to the toxicity in monolayer cultures is that the use of the live/dead stain to ascertain viability may under-count dead cells in the monolayer culture platform. Dead cells usually detach from the culture well surface, and as they are removed during the pipetting of the stain solutions, the process results in higher apparent viability due to under-representation of the dead cell population. In this work, all the cells were first removed from the culture well using trypsin/EDTA. The entire suspension containing both live and dead cells was then stained, centrifuged, and imaged in order to reduce the under-counting effect.

This platform, composed of a glass substrate 15 bonded to transparent PDMS microchannels 35 and chambers 45, permits image-based endpoint detection. A fluorescent dye-based assay is easily detected through this platform.

Dye exclusion assays such as the Live/Dead Invitrogen kit are rapid, and the reagents may be applied to microchannels 35 and chambers 45. Results from dye exclusion assays must take into account factors including the time required for cell membranes to rupture following exposure to cytotoxic agents. During this time, before the membrane is compromised, cells may remain metabolically active. In addition, dead cells will disintegrate, and living cells will proliferate, during this time. These factors may thus contribute to assays such as the Live/Dead stains giving different results than assays such as MTT, MTS, and Alamar Blue.

Microfluidic systems have applications in drug discovery and drug development processes, including sample preconcentration, separations, protein arrays, cellular interaction arrays, and cell-based high content screening. Three-dimensional (3-D) culture methods are used to study drug penetration in tumors. 3-D multicellular aggregates are used to simulate the tumor microenvironment in vivo and provide more complexity than a standard monolayer culture environment. Spheroids of tumor cells have been shown to have more resistance to doxorubicin than cells grown in monolayer or two-dimensional culture, and have been used in the evaluation of anticancer drugs. Small aggregates of 25-50 cells have shown more resistance to drugs and radiation treatment than monolayer cells. This resistance may be attributed to contact with the microenvironment, including cell-cell contacts and cell extracellular matrix contact.

Flow-focusing methods produce alginate droplets 105 with highly uniform diameters (coefficient of variation often is less than 5%). Alginate droplets 105 are generated through shear at the interface between two parallel streams. While not being bound by any theory, an explanation of uniform diameter droplets is the continuous phase places viscous stress on the immiscible dispersed phase, which is balanced by the surface tension. The viscous shear stress tends to extend the interface, while the competing surface tension effect tends to reduce the interfacial area. Droplets are created above a critical stress, and droplet formation is characterized by the dimensionless capillary number Ca=μv/γ, which gives the ratio of viscous forces to surface tension, where m is the viscosity of the continuous phase, v is the velocity of the droplet, and γ is the interfacial tension between the two phases. Droplet size is therefore a function of the fluid viscosities, surface tension, microfluidic channel geometry, and flow rates.

Alginate hydrogels may be used in cell encapsulation and release; examples include transplantation of insulin-producing pancreatic islet cells to treat diabetes, yeast cells in a lamellar geometry, tumor spheroids in lamellae, and mammalian cells in alginate droplets 105. Alginates are block copolymers which cross-link in the presence of divalent cations such as Ca²⁺. Microfluidic gelation of alginate gel beads 100 which encapsulate cells has been demonstrated using chaotic advection to mix the alginate precursor and calcium solution.

### EXAMPLES

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples.

### Example 1

### Preparation of a Droplet Formation Device

High aspect ratio features for microchannels and inlet/outlet reservoirs for a droplet formation structure were patterned using SU-8 photoresist on a silicon substrate. The droplet formation SU-8 photoresist on the silicon substrate served as a droplet formation mold master. Poly(dimethylsiloxane) (PDMS) (Sylgard, USA) was poured onto the droplet formation silicon mold master to make a droplet formation PDMS casting. A droplet formation plastic mold master was cast using a two-part polyurethane on the droplet formation PDMS casting. A droplet formation PDMS structure was cast from the droplet formation plastic mold master following a curing at about 60° C. for about two hours. The droplet formation PDMS structure was peeled off the droplet formation plastic mold master. The droplet formation PDMS structure was bonded onto a PDMS substrate. Access to the inlets 25 and outlets 30 were punched through the elastomer and fluidic interconnect was made using syringe needle tips 145.

### Example 2

### Preparation of a Cell Culture Chip Device 120

High aspect ratio features for microchannels and inlet/outlet reservoirs for a cell culture chip structure were patterned using SU-8 photoresist on a silicon substrate. The cell culture chip SU-8 photoresist on the silicon substrate serves as a cell culture chip silicon mold master. Poly(dimethylsiloxane) (PDMS) (Sylgard, USA) was poured onto the cell culture chip silicon mold master to make a cell culture chip PDMS casting. A cell culture chip plastic mold master was cast using a two-part polyurethane on the cell culture chip PDMS casting. A cell culture chip PDMS structure was cast from the cell culture chip plastic mold master following a curing at about 60° C. for about two hours. The cell culture chip PDMS structure was peeled off the cell culture chip plastic mold master. The droplet formation PDMS structure was bonded to a glass substrate 15, forming closed channels. Strong bonding was achieved by briefly treating the PDMS structure and the glass substrate in ozone (Jelight, USA). Access to the inlets 25 and outlets 30 were punched through the elastomer and fluidic interconnect was made using syringe needle tips 145.

### Example 3

### Alginate Precursor with LCC6/Her-2 Cell Suspension

LCC6/Her-2 breast tumor cells were maintained in Dulbecco's Modified Eagle Medium (“DMEM medium”) supplemented with 10% fetal bovine serum (FBS), 100 U/mL penicillin and 100 U/mL streptomycin. All cells were cultured in flasks for several days prior to microfluidic experiments. A 2.0 wt. % alginate solution was prepared using an LF120M type alginate mixed with Tris-HCl (50 mM, adjusted to pH 7.8 with HCl). The solution was passed through a 5.0 μm syringe filter to remove particulates. The 40 mM CaCl₂ solution was also buffered with 50 mM Tris-HCl, pH 7.8. All solutions were autoclaved before use. Cells were dissociated from culture flasks with 0.25% trypsin in phosphate buffered saline. Cell suspensions were prepared at a concentration of 10×10⁶ cell/mL using DMEM medium mixed with 2.0 wt. % alginate.

### Example 4

### LCC6/Her-2 Gelled Droplets

Gelled alginate droplets 100 (FIG. 7A) were generated from alginate droplets 105 that were prepared in the droplet formation chip 125. The formation device provided for the introduction of two dispersed phases and an immiscible solvent. The dispersed phases consisted of two solutions: the alginate precursor with the cell suspension of Example 3, and the calcium buffer. The immiscible solvent was n-hexadecane. All three solutions were injected into the channel 75 of the droplet formation chip 125 with mixing at the nozzle 80 using a pressure control system and a 2% concentration of Span 80 surfactant was used to stabilize the alginate droplets 105. The alginate droplets 105 were collected in a calcium salt bath to form alginate gel beads 100. The gelled alginate droplets 100 were washed in phosphate buffered saline, centrifuged, and re-suspended in culture media.

### Example 5

### LCC6/Her-2 Gelled Droplets Loaded onto Chip Device

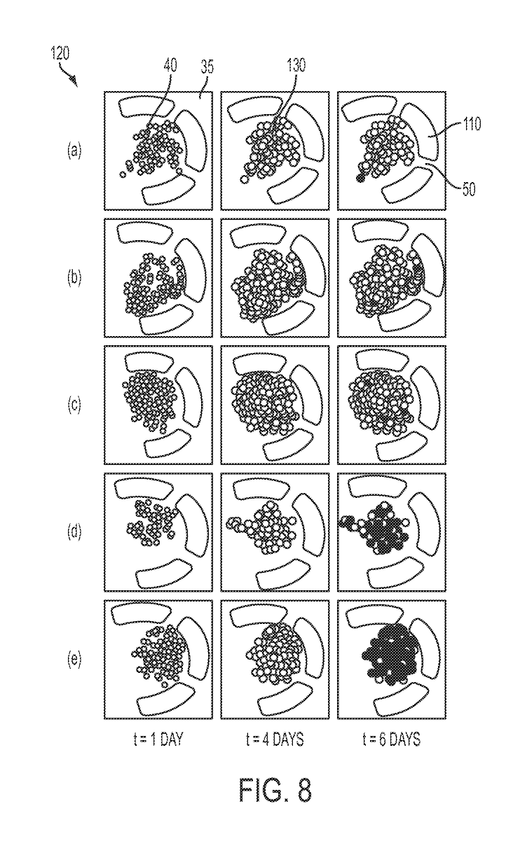

The alginate gel beads 100 of Example 4 were loaded into the microfluidic cell culture chip device 120, where they were trapped for cell culture (FIGS. 7B and 8). The loaded microfluidic cell culture chip device 120 was then placed into a standard 6-well plate, and the well plate was placed into an incubator with an atmosphere of 5% CO₂ and at 37° C. The microfluidic chips were connected to a syringe pump and DMEM culture medium was circulated at a rate of 0.25 μl min⁻¹.

As a first control, alginate gel beads containing cells were made by using a syringe with a 25 gauge needle to dispense droplets of the 2.0 wt. % alginate with cell suspension into a Ca²⁺ bath. The gelled control beads were then placed into the culture medium in a standard polystyrene well plate for incubation. As a second control, a two-dimensional, monolayer culture in standard multi-well plates was prepared.

### Example 6

### Treating of LCC6/Her-2 Gelled Droplets with Doxorubicin

Doxorubicin (Dox) is an anthracycline molecule that intercalates in DNA and inhibits topoisomerase II. As an anticancer agent, the drug inhibits RNA and DNA synthesis. During on-chip drug testing, the Dox solution was prepared with 0.2% dimethylsulfoxide (DMSO) and DMEM culture medium. After visual confirmation of spheroid formation at four days, the drug-free culture media was replaced with 400, 800, 1200, and 1600 nM Dox solutions. The drug solution was continuously perfused through the device at a rate of 0.25 μl min⁻¹ for two days. DMSO controls, in which the corresponding amount of DMSO in culture medium with no drug, were also carried out. Toxicity was examined after 48 h of drug dosing by quantifying cell viability.

### Example 7

### Assessment of Cell Viability

Cell viability was indicated with live/dead calcein AM/ethidium homodimer-1 stains (Invitrogen), which were applied through pressure-driven flow control to the cells while they were entrapped in alginate in the microdevice. Calcein AM (excitation 495 nm, emission 515 nm) was retained within live cells and EthD-1 (excitation 495 nm, emission 635 nm) was excluded by the intact plasma membrane of live cells. Live cells were identified by the presence of intracellular esterase activity, which turns the non-fluorescent cell-permeant calcein AM into fluorescent calcein. The ethidium homodimer had high binding affinity for nucleic acids. Since the molecule had four positive charges, it was excluded from living cells with intact membranes. Living cells showed green fluorescence color and the dead cell nuclei showed red fluorescence color. Here, 4 μM EthD-1 and 2.5 μM calcein AM in PBS was injected into the channel with a syringe and incubated for thirty minutes. The dyes diffused through the alginate to stain the cells embedded within.

All stained samples were imaged using fluorescence microscopy. The imaging system consisted of a fluorescent microscope (Nikon TE2000U) and a cooled, color CCD camera (Retiga). In each microfluidic chamber 45, scanning laser confocal images (488 nm and 543 nm excitation) were also acquired (NIS Elements, Nikon Instruments). Image processing was done using ImageJ. The number of living cells NG was calculated by counting the number of pixels in the green (living cells) channel in the confocal images and normalizing for the size of one cell. The number of dead cells NR was similarly calculated using the red pixels. The fluorescent stains were used to show the proportion and distribution of live and dead cells after drug treatment for two days.

The survival rate was calculated as N_{G}/(N_{G}+N_{R}). The proliferation rate is calculated as (N₄−N₁)/N₁ before drug treatment, and as (N₆−N₄)/N₄ after drug treatment, where Nₓ is the number of cells on the xth day.

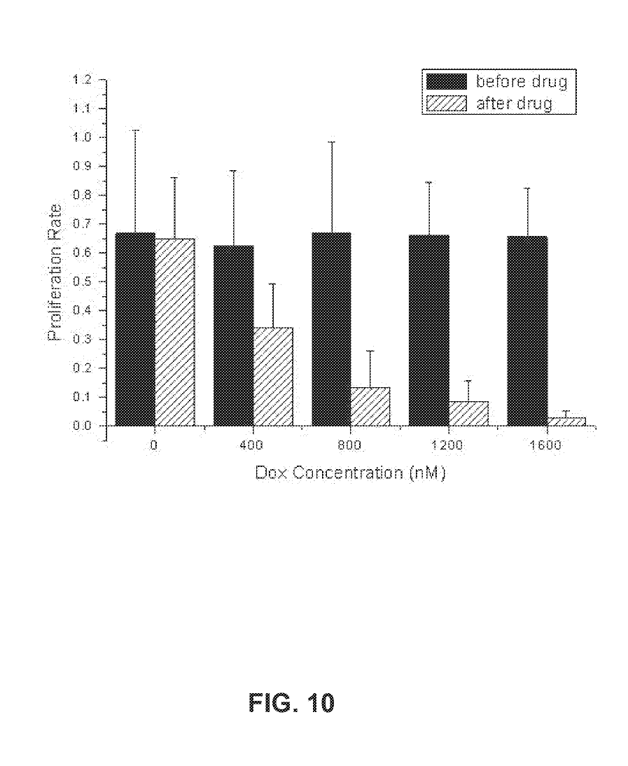

For LCC6/Her2 cells cultured within alginate gel beads 100, cell activity as measured using the standard MTS assay was 35%, while cell viability as measured using the Live/Dead stains was 83%, in both cases after 48 h treatment with 800 nM doxorubicin. The proliferation data (FIG. 10), which account for the total number of cells, also show this difference, with a marked proliferation decrease at 1600 nM Dox exposure compared to only a 20% viability decrease at that dosage. Thus, the absolute number of surviving cells, in addition to the percentage of living or dead cells, may be an important parameter in drug screening. This can be obtained by processing the data from image-based high-throughput screening systems.

### Example 8

### Integration of Droplet Formation, Droplet Gelation, and Cell Culture on One Chip

When the droplet formation and microsieve traps are in series on the same chip, residual hexadecane in the chip may have difficulty of removal using moderate flow rates to flush it out after droplet formation. High flow rates compress and damage the alginate beads collected within the chip. Thus, separation of the droplet formation chip 125 and cell culture chip device 120 permitted the cell culture to remain free of hexadecane.

### Example 9

### On-Chip Tumor Cell Culture

Alginate beads were gelled to encapsulate breast tumor cells. After the alginate gel beads 100 were trapped in microsieve structures 110, the cells were cultured for several days to permit spheroid 130 formation. The three-dimensional environment permitted the cells to form multicellular aggregates, which is not observed in traditional monolayer culture. Using this platform, the dose-dependent cytotoxic effect of doxorubicin was measured. Increasing doxorubicin concentration decreased viability and proliferation. Multicellular resistance was observed at 1200 and 1600 nM doxorubicin, with spheroids 130 having higher viability than cells in traditional monolayer culture. The location of each alginate gel bead 100 was maintained in the same position within the cell culture chip device 120, so that differences in cell proliferation and drug response between spheroids were monitored and tracked.

### Example 10

### On-Chip Tumor Cell Culture

The LCC6 (parental line MDA-MB-435) cell line is an ascites model of human breast cancer. Ascites tumor cells typically grow as a cell suspension in the peritoneal fluid. The ascites are formed when solid tumors shed cells into the peritoneal cavity. Cells were used from a LCC6 line which were permanently transfected with the Her2 gene. After encapsulation, the cells were randomly distributed throughout the alginate gel beads 100. As a non-adhesive hydrogel, the alginate allowed the cells to proliferate and form multicellular spheroids. The FIG. 7B images show dispersed, individual tumor cells maintained intact cell membranes. FIG. 7A shows images of alginate gel beads 100 immediately after droplet formation. These alginate gel beads 100 were suspended in a Petri dish. Each bead is round and the edge 140 of the alginate is very clear before the beads are loaded into the microchannel 35. The tumor cells gradually formed small aggregates within the alginate gel beads after 4 days culture. FIG. 7B shows images of alginate gel beads after 4 days culture in the microsieve structures 110. The dispersed cells have proliferated and formed multicellular aggregates as spheroids 130. Scale bars for FIGS. 7A and 7B: 100 μm.

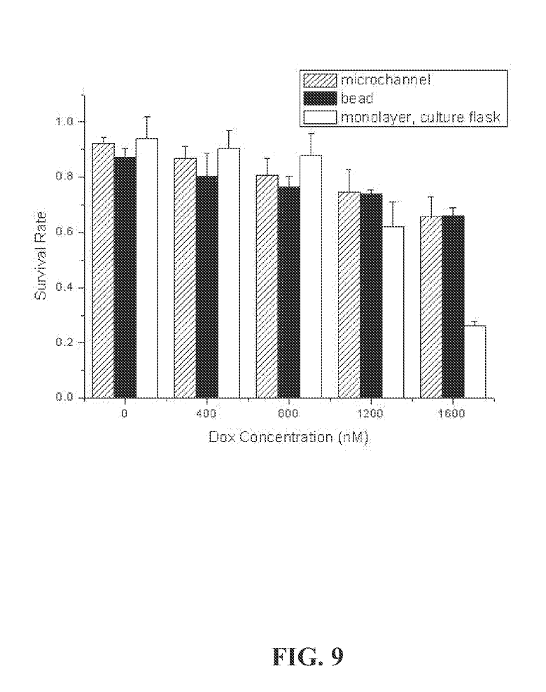

Images from confocal microscopy were used to determine cell survival rate and proliferation inside the three dimensional multicellular aggregates after exposure to different doxorubicin concentrations. FIG. 8 shows images of LCC6/Her2 breast tumor cells proliferating and forming multicellular spheroids while encapsulated in alginate gel beads 100. Spheroid 130 formation was visually confirmed four days after cell seeding. Doxorubicin was the perfused with (a) 0, (b) 400, (c) 800, (d) 1200, and (e) 1600 nM doxorubicin for two days, and cell viability was measured at the end of that period after staining with a live/dead viability kit and confocal imaging. Images were selected out of the confocal stack to avoid overlapping of the same cells between images. The total on-chip culture period, including exposure to doxorubicin, was six days. The results show a dose-dependent decrease in survival rate (FIG. 9) as well as proliferation rate (FIG. 10). FIG. 9 shows the effects of doxorubicin concentration on the cell survival rate in various culture environments. The hashed bar shows microchannel: small tumor spheroids encapsulated in alginate gel beads in a microchannel; the black bar shows bead: tumor spheroids encapsulated in alginate gel beads and suspended in a culture flask; and the white bars show a monolayer: standard culture flask. Five groups of cells, treated with 0, 400, 800, 1200, 1600 nM doxorubicin respectively, were investigated. Cells were stained using the live/dead assay. The number of living cells N_{G} was calculated by counting the number of pixels (living cells) channel in the confocal image and normalizing for the size of one cell. The number of dead cells N_{R} was similarly calculated. FIG. 10 shows the effects of doxorubicin concentration on the cell proliferation rate of five groups of tumor spheroids before drug treatment (black bars, cultured 4 days on-chip) and after drug treatment for 2 days (hashed bars). The proliferation rate is calculated as (N₄−N₁)/N₁ before drug treatment, and as (N₆−N₄)/N₄ after drug treatment, where Nₓ is the number of cells on the x^{th} day.

In each case, the cell response within alginate gel beads made by syringe and cultured in a standard culture flask (“bead”) were compared to alginate gel beads in microchannels (“microchannel”) and cells in standard monolayer culture in the flasks (“monolayer, culture flask”). The “bead” and “microchannel” cells were in both cases exposed to the three-dimensional alginate culture environment, and differed in the presence of the hexadecane during droplet formation and the use of microfluidic channel during cell culture. This simple viability assay did not indicate any additional toxicity effects, at a basic level, from hexadecane or the PDMS microchannel material, or effects of perfusion flow as opposed to static media, as indicated by the similar survival rates for the “bead” and “microchannel” cases. Thus, the “bead” was a control which can assist in illustrating the utility of microfluidic platforms for cell encapsulation and culture.

The results also showed that spheroids of tumor cells have more resistance to doxorubicin than cells grown in monolayer or two-dimensional culture (FIG. 9). The multicellular resistance index, defined as the ratio [IC₅₀, spheroid/IC₅₀, monolayer], can range from 35 for doxorubicin to 6625 for vinblastine on A549 human lung cells. Multicellular resistance was also demonstrated in human MCF-7 breast tumor cells encapsulated in alginate-poly-L-lysine-alginate microcapsules, with lower inhibition rates in multicellular spheroids than in monolayers for cells treated with mitomycin C, adriamycin (trade name for doxorubicin), and 5-fluorouracil as determined by the MTT assay. Spheroids of EMT-6 mammary sarcoma cells also demonstrated higher resistance to different exposure doses of adriamycin than monolayer cells, with spheroids created in a spinner flask.

### Example 11

### On-chip Tumor Cell Culture

The present application provides a droplet-based microfluidic system for formation of alginate gel beads 100 for cell encapsulation and 3-D culture. The cell culture platform allows continuous flow control for both long-term cell culture as well as drug testing. An example of two separate chips is shown in FIGS. 1/2 and 3/4. Two separate chips may be used, one for droplet formation 125 and a separate chip for cell culture 120. Channels 75 in the droplet formation chip 125 were 113 micrometers in depth, 400 μm in width in the main channel 75, and 100 μm in width at the nozzle 80. Each cell culture chip device 120 has two chambers 45. Each chamber 45 contains 14 microsieves 110 for alginate droplet trapping. Each microsieve 110 is semicircular with two apertures (48 μm width) 50 to facilitate bead trapping. An alginate gel bead 100 may contain one or more alginate encapsulated cells.

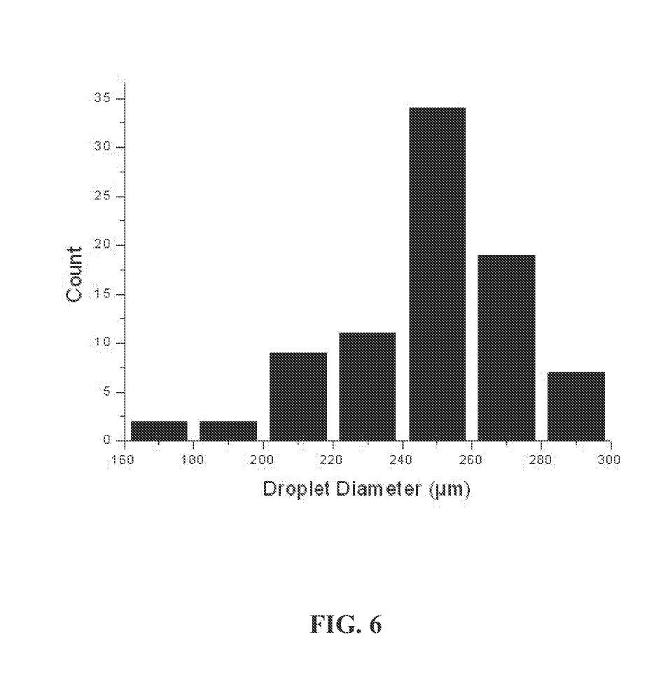

One approach uses an off-chip calcium ion bath for gelation of alginate droplets 105 formed using shear flows in a microfluidic chip. After rinsing in culture media to remove the hexadecane, the alginate gel beads 100 were loaded into the cell culture chip device 120 containing traps as microsieves 110. An example is shown in FIG. 5 where each microsieve 110 was semicircular with an inner diameter of 300 μm, with two apertures (48 μm width) 50 which permitted the culture medium to flow through the microsieve 110 during bead loading. The apertures 50 reduced flow resistance and facilitated bead trapping. Each microsieve 110 contains one alginate gel bead 100, and each alginate gel bead 100 contains approximately 100 cells on the day of cell loading on the chip. The channels were 113 μm in depth and each microsieve 110 is semicircular with an inner diameter of 300 μm. The scale bar in FIG. 5 is 200 μm. The average bead diameter was 251 μm, with 10% coefficient of variation (FIG. 6), standard deviation 27.25, n=84.

### Example 12

### On-Chip Tumor Cell Culture

As stated above, two separate chips may be used, a droplet formation chip 125 and a separate cell culture chip device 120. By avoiding the acidic environment and by using off-chip gelation, cell viability was maintained above 90% in the alginate gel beads 100 [viability calculated after 6 days culture in the microchannel as NG/(NG+NR), where NG was calculated by counting the number of pixels in the green (living cells) channel in the confocal images and normalizing for the size of one cell and NR was similarly calculated using the red pixels (dead cells)]. Hexadecane is highly immiscible with water and has low solubility (9.0×10⁻⁸ g/100 g water at 25° C.) in the aqueous phase, allowing high cell viability in alginate gel beads 100 formed in hexadecane.

What is claimed is:

1. A method of isolating at least one cell of interest, the method comprising:
disposing a collection of hydrogel encapsulated cells on a surface to prepare a fixed array; and
assaying the array to identify at least one hydrogel encapsulated cell of interest.

2. The method of claim 1, further comprising removing the at least one hydrogel encapsulated cell of interest from the array to provide an isolated hydrogel encapsulated cell.

3. The method of claim 2, further comprising releasing the isolated hydrogel encapsulated cell to form a released non-encapsulated cell.

4. The method of claim 3, further comprising harvesting the released non-encapsulated cell.

5. The method of claim 3, further comprising culturing the released non-encapsulated cell.

6. The method of claim 1, wherein the collection of hydrogel encapsulated cells comprises a hydrogel selected from alginate, collagen, and a protein mixture secreted by mouse sarcoma cells, or any combination thereof.

7. The method of claim 1, wherein the surface is a microfluidic chip.

8. The method of claim 6, wherein the collection of alginate encapsulated cells is prepared by:
mixing alginate precursor and at least one cell in an immiscible solvent to form a dispersed phase; and
gelling the dispersed phase using a calcium ion bath to provide the collection of alginate encapsulated cells.

9. The method of claim 8, wherein the immiscible solvent is selected from hexadecane, dodecane, toluene, benzene, decalin, octanol, silicone oil, vegetable oil, and fluorinated oil, or any combination thereof.

10. The method of claim 6, wherein releasing an isolated alginate encapsulated cell comprises de-crosslinking the alginate using a chelator.

11. The method of claim 10, wherein the chelator is selected from 2,2′-bipyridyl, dimercaptopropanol, ethylenediaminotetraacetic acid (EDTA), ethylene glycol-bis-(2-aminoethyl)-N,N,N′,N′-tetraacetic acid (EGTA), ionophores, nitrilotriacetic acid, NTA ortho-phenanthroline, gramicidin, monensin, valinomycin, salicylic acid, triethanolamine (TEA), polysaccharides, organic acids with at least two coordination groups, lipids, steroids, amino acids, peptides, phosphates, phosphonates, nucleotides, tetrapyrrols, ferrioxamines, and phenolics, or any combination thereof.

12. The method of claim 6, wherein releasing the isolated hydrogel encapsulated cell comprises using a protease.

13. The method of claim 12, wherein the protease is selected from dispase, trypsin, chymotrypsin, elastase, cathepsins, bromelain, actimidain, calpain, caspase, papain, mir1-CP, chymosin, rennin, pepsin, plasmepsin, nepenthesin, and collagenase, or any combination thereof.

14. The method of claim 1, further comprising incubating the fixed array.

15. The method of claim 1, wherein the cell is selected from a tumor cell, cancer stem cell, epithelial cell, diseased cell, and normal cell, or any combination thereof.

16. A method of making a fixed array, the method comprising:
mixing alginate precursor and at least one cell in an immiscible solvent to form a dispersed phase;
gelling the dispersed phase using calcium ions to form at least one alginate encapsulated cell; and
disposing the alginate encapsulated cell onto a surface to prepare a fixed array.

17. The method of claim 16, wherein the immiscible solvent is selected from hexadecane, dodecane, toluene, benzene, decalin, octanol, silicone oil, vegetable oil, and fluorinated oil, or any combination thereof.

18. The method of claim 16, further comprising allowing the cell to proliferate within the alginate encapsulated gel.

19. The method of claim 16, further comprising culturing the at least one cell before mixing with the alginate precursor.

20. The method of claim 16, further comprising washing the alginate encapsulated cell before disposing the alginate encapsulated cell.

21. The method of claim 20, further comprising centrifuging the washed alginate encapsulated cell.

22. The method of claim 21, further comprising suspending the centrifuged alginate encapsulated cell.

23. An array comprising a glass substrate bonded to a patterned siloxane structure having inlets, outlets and microchannels.

24. The array of claim 23, further comprising a collection of alginate encapsulated cells trapped in the microchannels.

25. The array of claim 23, wherein the patterned siloxane structure comprises at least one chamber having the microchannels.

26. The array of claim 23, wherein the microchannels comprise sieves, weirs, cavities, or wells, or any combination thereof.

27. The array of claim 23, wherein the patterned siloxane structure comprises at least one aperture to facilitate trapping of an alginate encapsulated cell.

28. The array of claim 23, wherein the patterned siloxane structure comprises a material selected from poly-(dimethylsiloxane), polyurethane, polystyrene, parylene, and polyimide, or any combination thereof.

29. The array of claim 23, wherein the patterned siloxane structure is transparent.

30. An array kit comprising:
a glass substrate; and
a patterned siloxane structure having microchannels, inlets and outlets.

31. The kit of claim 30, further comprising a hydrogel to encapsulate cells.

32. The kit of claim 31, wherein the hydrogel may be selected from alginate, collagen, and Matrigel™, or any combination thereof.

33. The kit of claim 30, wherein the patterned siloxane structure comprises a material selected from poly-(dimethylsiloxane), polyurethane, polystyrene, parylene, and polyimide, or any combination thereof.

34. The kit of claim 30, wherein the patterned siloxane structure is transparent.

35. The kit of claim 30, further comprising a siloxane substrate.

36. The kit of claim 30, further comprising a siloxane droplet formation structure having at least one channel and a nozzle.

37. An array kit comprising a glass substrate; a cell culture mold comprising microchannels, inlets and outlets; a droplet formation mold having at least one channel and a nozzle; and a siloxane substrate.

38. The kit of claim 37, wherein the siloxane substrate comprises a material selected from poly-(dimethylsiloxane), polyurethane, polystyrene, parylene, and polyimide, or any combination thereof.

39. A method of making a microfluidic apparatus, the method comprising:
applying a layer of photoresist to a silicon substrate to make a silicon mold;
pouring a layer of siloxane into the silicon mold to make a patterned siloxane structure;
bonding the patterned siloxane structure to a glass substrate to form a cell culture structure;
forming a droplet formation mold comprising at least one channel and a nozzle;
pouring a layer of siloxane into the droplet formation mold to make a siloxane droplet formation structure; and
bonding the siloxane droplet formation structure to a siloxane substrate to form a droplet formation structure.

40. The method of claim 39, further comprising treating the cell culture structure in ozone to achieve strong bonding between the glass substrate and the patterned siloxane structure.

41. The method of claim 39, further comprising treating the cell culture structure in ozone to achieve strong bonding between the glass substrate and the patterned siloxane structure.

42. The method of claim 39, wherein the patterned siloxane structure comprises microchannels, inlets and outlets.

43. The method of claim 42, further comprising making holes in the microfluidic apparatus to allow access to the inlets and outlets.

44. The method of claim 39, further comprising curing the patterned siloxane structure before bonding.

45. The method of claim 39, further comprising curing the siloxane droplet formation structure before bonding.

46. The method of claim 39, wherein the cell culture structure comprises sieves, weirs, cavities, and wells, or any combination thereof.

47. A method of making a fixed array, the method comprising:
incubating a cell suspended in a hydrogel in a buffer or medium to form a hydrogel encapsulated cell; and
disposing the hydrogel encapsulated cell onto a surface to prepare a fixed array.

48. The method of claim 47, wherein the hydrogel is collagen, or a protein mixture secreted by mouse sarcoma cells, or a combination thereof.

49. The method of claim 47, wherein the suspended cell is incubated at a temperature of at least about 25° C.

50. The method of claim 47, wherein the cell is suspended in hydrogel at a temperature of less than about 25° C.
